(19) United States
(12) Patent Application Publication
(10) Pub. No.: US 2002/0068100 A1
(43) Pub. Date: Jun. 6, 2002

(54) Combination therapy for premenstrual symptoms

(75) Inventors: Rakesh Kapoor, Saskatoon (CA); Alessandra Berruti, London (CA)

Correspondence Address: Alice O. Carroll, Esq., HAMILTON, BROOK, SMITH & REYNOLDS,P.C., 530 Virginia Road, P.O. Box 9133, Concord, MA, 01742-9133, US

(73) Assignee: Bioriginal Food & Science Corporation, Saskatoon (CA)

(21) Appl. No.: 09/975,048

(22) Filed: Oct. 11, 2001

Related U.S. Application Data
(60) Provisional application No. 60/239,790, filed on Oct. 12, 2000.

(51) Int. Cl. A61K 35/78; A61K 31/20; A61K 31/355

(52) U.S. Cl. 424/725; 424/736; 424/769; 424/775; 424/777; 514/558; 514/458

(57) ABSTRACT

Disclosed is a composition comprising ingredients a-f: a) n-6 fatty acid; b) n-3 fatty acid; c) vitamin E; d) extract of Viburnum opulus bark; e) extract of Vitex agnus castus berry; and f) bioflavonoid(s). Also disclosed is a method of treating premenstrual symptoms in a subject in need of such treatment and a method of treating irritable bowel syndrome or interstitial cystitis in a subject in need of such treatment. The method comprises the step of administering to the subject a therapeutically effective amount of the ingredients a-f, described above. Preferably, the subject is administered a composition comprising ingredients a-f.

### RELATED APPLICATIONS

This applications claims the benefit of U.S. Provisional Application 60/239,790, filed Oct. 12, 2000, the entire teachings of which are incorporated herein by reference.

### BACKGROUND OF THE INVENTION

Premenstrual syndrome (PMS) is a recurrent cyclic disorder associated with the cyclic hormonal rhythms of the menstrual cycle (Clin. Obstet. Gynecol. 40:564 (1997)). A large number of symptoms have been associated with PMS that are divided into physical, behavioral, and emotional symptoms. PMS may be associated with dysmenorrhea and other menstrual irregularities. Physical symptoms include bloating, abdominal and back cramps and discomfort, change in appetite, weight gain, breast tenderness and pain, and headache. Behavioral changes include anxiety, depression, lethargy, hypersomnia or insomnia, moodiness, irritability, anger, and social withdrawal. These symptoms vary in intensity from mild to severe and affect up to 90% of the women. About 5% of North American women suffer from moderate to severe symptoms affecting their daily life activities.

Pharmacologically active agents have been used to treat PMS and include antidepressants of the group belonging to selective serotonin reuptake inhibitors (SSRI's), anti-inflammatory agents including non-steroidal anti-inflammatory agents, anxiolytics, hormones (progesterone), dopamine agonists, and diuretics. However, drug treatments can have many disadvantages including side-effects and cost, and are not always effective.

As an alternative treatment for PMS, many patients have turned to plant based treatments and/or homeopathic remedies. One such example is the extract from the fruit of Vitex Agnis castis L. (see, for example, Lauritzen et al., Phytomedicine 4:183 (1997), Russo and Galletti, Acta Hort. 426:105 (1996) and Milewicz, et al., Arneimittelforschung 43:752 (1993)). Unfortunately, this remedy according to a number of reports is also accompanied by side-effects. For example, in a clinical trial involving 85 patients treated with extract of Vitex Agnis castis L., between five to twelve reported adverse events such as persistent gastroenteritis, nausea, allergic rashes, headache and acneiform facial inflammation (see pages 186-7 of Lauritzen et al., supra). In another study of twenty volunteers, adverse events were reported by thirteen patients, although a connection with the test medicine was evaluated as uncertain. Symptoms included skin reactions, dry mouth, disturbed sleep or tachycardia and gastrointestinal disorders (see Merz et al., Exp. Clin. Endocrinol. Diabetes 104:447 (1996)). Another reported shortcoming of Vitex Agnus castis L. is that patients can experience a long delay before symptoms are relieved. For example, Armann, Zeitschrift for Allgemeinmedizin 55:48 (1979) reported that six months can pass before the medicine takes full effect. Christie and Walker, The European Journal of Herbal Medicine 3:29 (1997) reported the symptoms of 68.5% of patients responded in 4-8 weeks after beginning treatment with Vitex Agnus castis L. for PMS.

As a consequence, there is a clear need for treatments for PMS having a rapid onset of action and reduced side-effects.

### SUMMARY OF THE INVENTION

A combination therapy for premenstrual symptoms has now been discovered which causes minimal side effects and rapidly relieves the symptoms associated with the condition. Specifically, 46 patients in a clinical trial involving 74 women reported a 50% or greater reduction in global scores measuring the severity of PMS symptoms within one menstrual cycle (Example 1) after using the combination therapy. The number of such patients increased to 78% and 85% in the second and third cycle, respectively (Example 1). Only four adverse reactions were reported, none of which could be connected with the combination therapy (Example 1). The combination therapy also relieved the symptoms of a patient with heritable Bowel Syndrome and Interstitial Cystitis (Example 2). Based on this discovery, a novel combination composition, a composition suitable for oral administration, a method of treating PMS, a method of treating Irritable Bowel Syndrome and a method of treating Interstitial Cystitis are disclosed herein.

One embodiment of the present invention is a composition. The composition comprises the following ingredients a-f: a) n-6 fatty acid or a source thereof; b) n-3 fatty acid or a source thereof, c) vitamin E; d) extract of Viburnum opulus bark; e) extract of Vitex Agnus castus berry; and f) citrus bioflavonoid(s). Optionally, the composition also comprises vitamin B6 and/or a physiologically acceptable salt of zinc.

Another embodiment of the present invention is a composition for oral administration to a subject. The composition comprises a physiologically acceptable carrier (e.g., a gelatin capsule, foodstuff or beverage) and a therapeutically effective amount of the ingredients a-f, described above. Optionally, the composition also comprises vitamin B6 and/or a physiologically acceptable salt of zinc.

Another embodiment of the present invention is a method of treating premenstrual symptoms in a subject in need of such treatment. The method comprises the step of administering to the subject a therapeutically effective amount of the ingredients a-f, described above. Preferably, the subject is administered a composition comprising ingredients a-f.

Yet another embodiment of the present invention is a method of treating irritable bowel syndrome or interstitial cystitis in a subject in need of such treatment. The method comprises the step of administering to the subject a therapeutically effective amount of the ingredients a-f, described above. Preferably, the subject is administered a composition comprising ingredients a-f.

The combination therapy of the present invention provides rapid relief from the symptoms of PMS, typically within one menstrual cycle. In addition, it may be effective in relieving of the symptoms of other conditions, such as Irritable Bowel Syndrome and Interstitial Cystitis. The combination therapy provides relief from the symptoms of these conditions while causing few if any side-effects.

### DETAILED DESCRIPTION OF THE INVENTION

The present invention is directed to a combination composition which can be used to treat or relieve the symptoms of PMS, Irritable Bowel Syndrome or Interstitial Cystitis. These combination compositions can be provided in bulk form, in a multidosage form or in a unit dosage form. Optionally, these combination compositions can also include other active ingredients such as vitamins (e.g., vitamin B6) or physiologically acceptable salts of zinc. The compositions are typically administered orally and therefore often additionally comprise a physiologically acceptable carrier.

In a preferred embodiment, ingredients a-f are present in the compositions of the present invention in the following amounts:

a) between about 45 to about 1000 parts of the total combined weight of ingredients a-f is n-6 fatty acid;

b) between about 50 to about 1000 parts of the total combined weight of ingredients a-f is n-3 fatty acid;

c) between about 10 to about 90 parts of the total combined weight of ingredients a-f is vitamin E;

d) between about 50 to about 600 parts of the total combined weight of ingredients a-f is extract of Viburnum opulus bark;

e) between about 25 to about 300 parts of the total combined weight of ingredients a-f is extract of Vitex agnus castus berry; and

f) between about 5 and about 100 parts of the total combined weight of ingredients a-f is citrus bioflavonoid(s).

As used herein, the parts of the total combined weight of ingredients a-f that are a particular ingredient is the weight of the ingredient relative to the total combined weight of ingredient a-f. For example, if the composition contains 300 mg n-6 fatty acid, 400 mg n-3 fatty acid, 60 mg vitamin E, 150 mg extract of Viburnum opulus, 75 mg extract of Vitex agnus castus and 50 mg citrus bioflavonoid(s), then 300 parts of the total combined weight of ingredients a-f is n-6 fatty acid; the total combined weight of ingredients a-f would be 1035 mg. Similarly, if the units of the weights recited in the previous sentence were kilograms instead of milligrams, then n-6 fatty acid would still represent 300 parts of total combined weight of ingredients a-f, the total combined weight of ingredients a-f would now be 1035 kg.

In a more preferred embodiment, ingredients a-f are present in the compositions of the present invention in the following amounts:

a) between about 200 to about 400 parts of the total combined weight of ingredients a-f is n-6 fatty acid, preferably gamma linolenic acid, (GLA);

b) between about 200 to about 500 parts of the total combined weight of ingredients a-f is n-3 fatty acid, preferably alpha linolenic acid (ALA);

c) between about 30 to about 90 parts of the total combined weight of ingredients a-f is vitamin E;

d) between about 100 to about 300 parts of the total combined weight of ingredients a-f is extract of Viburnum opulus bark;

e) between about 50 to about 150 parts of the total combined weight of ingredients a-f is Vitex agnus castus berry; and

f) between about 15 to about 75 parts of the total combined weight of ingredients a-f is citrus bioflavonoid(s).

n-6 fatty acids include gamma-linolenic acid (hereinafter “GLA”) and dihomo-gamma linoleic acid. GLA is a preferred n-6 fatty acid for use in the compositions described herein. The compositions of the present invention can comprise substantially pure n-6 fatty acid or, alternatively, n-6 fatty acid in a form that is partially isolated from one or more natural sources. For example, the compositions can comprise Borage oil (obtained from the seeds of Borago officinalis L.), seed oils of Evening Primrose (Oenothera biennis L.) or Black Current (Ribes nigrum L.) or oil from Algae (Tetrahymena sp.) as the source of GLA or GLA in a partially or substantially purified from any of these natural sources. Alternatively, GLA can be supplied as a mixture of partially purified forms or a mixture of substantially purified and partially purified forms. n-3 fatty acids include alpha linolenic acid (hereinafter “ALA”) and metabolites of alpha linolenic acid. Examples of metabolites of ALA include stearidonic acid (C18:4), eicosapentaenoic acid (EPA, C20:5) and docosahexaenoic acid (DHA, C22:6). ALA is a preferred n-3 fatty acid for use in the compositions described herein. The compositions of the present invention can comprise substantially pure ALA, or, alternatively, an n-3 fatty acid in a form that is partially isolated from natural sources. For example, the compositions can comprise flax oil (obtained from the seeds of Linum usitatissimum L.) or Perilla oil (obtained from the seeds of Perilla frutescence), as the source of ALA or a partially or substantially purified form of ALA from any of these natural sources. Alternatively, ALA can be supplied as a mixture of partially purified forms or a mixture of substantially and partially purified forms.

Preferably, the ratio of n-6 fatty acid to n-3 fatty acid in the compositions of the present invention is about 1.75 to about 4.5 weight/weight (w/w) and more preferably about 1.90 to about 2.75 w/w. When the compositions of the present invention comprise ALA and GLA, the ratio of ALA to GLA in the composition is preferably between about 1.0 to about 2.0 w/w, more preferably between about 1.2-1.7 w/w.

Vitamin E can be in the form of d-alpha tocopherol or d-alpha tocopheryl acetate or mixed tocopherols or racemic (synthetic) tocopherols or tocotrienols. Vitamin E is commercially available, for example, from Archer Daniels Midland Co. (Toronto, ON, Canada), Hoffman-LaRoche (Cambridge, ON, Canada) and Cognis Canada (Mississauga, ON, Canada). Alternatively, the composition can comprise mixtures of these forms of vitamin E.

An “extract” is the material which dissolves in a solvent after contacting a plant substance with a suitable solvent. Preferably, the plant substance is contacted for a sufficiently long period of time and at a suitable temperature so that substantially all of the soluble material is removed by the solvent. The solvent together with the dissolved soluble plant material is referred to as the “liquid extract”. The term “extract” refers to the soluble plant material remaining after removal of the solvent. The solvent can be removed by any suitable means, including evaporation, lyophilization, spray drying and the like.

Extract of Vitex agnus castus L. fruits (berries) refers to an extract of the fresh or dried berries of Vitex agnus castus. Extract of Viburnum opulus L. bark refers to an extract of the bark of Viburnum opulus or Viburnum prunifolium. Suitable solvents for the extraction of either of these plant materials include, but are not limited to alcohol, water, a mixture of alcohol and water, hexane, supercritical carbon dioxide. Typically, fresh or dried berries (or fresh or dried bark) are powdered to facilitate the extraction. The powdered (fine to coarse) berries (or bark) are then extracted with alcohol or water (typically from about four to seven parts solvent per part berry w/w) or a mixture of alcohol and water at a temperature between about 15° C. to about 100° C. The liquid extract so obtained is concentrated under reduced pressure to remove the alcohol. The de-alcoholized extract can be spray dried or freeze dried with or without a carrier such as starch, modified starch, vegetable gums, cyclodextrins or maltodextrins. Alternatively, the solvent of extraction can be hexane, dichloromethane, diethyl ether or supercritical carbon dioxide. One kilogram of Vitex agnus castus extract (or Viburnium opulus L. extract) can typically be prepared from 4 to 15 kg of fresh or dried berries (or bark). Extract of Vitex agnus castus L. can also be obtained commercially from, for example, Infinity Industries, Inc. (Ronkonkoma, N.Y.), DNP International (Markham, ON, Canada), Gourmet Nutrition (Ste-Julie, QB, Canada) and Stryka Botanical Co., Inc. (Somervilee, N.J.) ; and extract of Viburnum opulus L. can be obtained commercially from, for example, Ashland Chemical Company (Irvine, Calif.), ExtractPlus Inc. (Vista, Calif.) and Gourmet Nutrition (Ste-Julie, QB, Canada).

Bioflavonoids are naturally occurring phytochemicals that are derivatives of coumarin or flavone. Examples include hepseridine, neohesperidine, naringin, rutoside, sinensetin, nobiletin, tangeretin and the like. Typically, mixtures of bioflavonoids are used in the compositions of the present invention, preferably obtained from extraction of the rind of Citrus fruits (orange, grape fruit, lemon or a mixture of these fruits). Bioflavonoids can be extracted from these sources using procedures similar to those described above for Vitex agnus castus or Viburnum opulus L. Alternatively, citrus bioflavonoids can be obtained commercially from, for example, Global Marketing (Hayward, Calif.), P. L. Thomas and Co., Inc. (Morristown, N.J.), JSZ International Inc. (Rowland Heights, Calif.), QBI, (Quality Botanical) (South Planifield, N.J.) and Gourmet Nutrition (Ste-Julie, QB, Canada).

“Bulk form” refers large quantities of the composition, typically quantities greater than would be sold to an end-user. Thus, “bulk form” refers to quantities produced by a manufacturing process or subdivided portions of such quantities which are suitable for sale to intermediate vendors or parties who package, formulate or otherwise process but do not consume the composition.

A unit dosage form contains the amount of each ingredient which is to be administered to a subject at a given point in time. A unit dosage form can be a pill, capsule (e.g., soft or hard gelatin), powder, solution, tablets, emulsions, suspensions or other suitable pharmaceutical formulation. Other standard pharmaceutical formulations are described in Remington's Pharmaceutical Sciences, Mack Publishing Company, Easton, Pa. Alternatively, the unit dosage form can be a foodstuff, including nutritional bars, beverages, drink mixes, jellies and the like.

A “multidosage form” is typically sold to the end user, but comprises more than one dosage form. Therefore, a multidosage form is typically smaller than a bulk form, but requires the end user to divide the composition into a suitable unit dosage form. For example, the composition may be sold as a powder containing more than one unit dose of active ingredients, requiring the subject to measure the appropriate dose by, for example, weight or volume. Other forms suitable for subdivision include solutions and foodstuffs which contain more than one unit dose of active ingredients. The term “multidosage form” also includes a container (e.g., a bottle) comprising a multiplicity of unit dosage forms of the composition, for example, a multiplicity of capsules, tablets and the like.

As mentioned above, bulk, multidosage and unit dosage forms often additionally comprise a physiologically acceptable carrier. Suitable carriers therefore include sterile/purified water, hard gelatin, soft gelatin, cyclodextran, starch, modified starch, vegetable gums, cyclodextrins, maltodextrins or foodstuffs.

Premenstrual Syndrome is a complex of physical and emotional changes occurring several days before the onset of menstrual flow. Symptoms include depression, anxiety, anger, feelings of hopelessness, feelings of being alone, lessened ability to concentrate, cravings, fatigue, bloating, cramps, gastrointestinal irregularities, breast tenderness, headaches, loss of sleep and hot flashes.

Irritable Bowel Syndrome is a condition characterized by disordered gastrointestinal motility of unknown cause. Symptoms include abdominal pain and constipation, diarrhea or both alternating.

Interstitial cystitis is a persistent and chronic condition characterized by a non-bacterial inflammation of unknown cause involving mucosa and muscularis of the bladder. Symptoms include bladder fullness, frequency, small urine volume and lower abdominal pain.

“Therapeutically effective amount” is the quantity which results in the amelioration or improvement of one or more symptoms of the condition being treated. Preferably, therapeutically effective amounts of each ingredient include the following:

a) between about 45 to about 1000 mg per day of n-6 fatty acid;

b) between about 50 to about 1000 mg per day of the n-3 fatty acid;

c) between about 10 to about 90 mg per day of vitamin E;

d) between about 50 to about 600 mg per day of extract of Viburnum opulus bark;

e) between about 25 to about 300 mg per day of extract of Vitex agnus castus berry; and

f) between about 5 and about 100 parts mg per day of citrus bioflavonoid(s).

In a preferred embodiment, therapeutically effective amounts of each ingredient include the following:

a) between about 200 to about 400 mg per day of gamma linolenic acid, (GLA);

b) between about 200 to about 500 mg per day of alpha linolenic acid (ALA);

c) between about 30 to about 90 mg per day of vitamin E;

d) between about 100 to about 300 mg per day of extract of Viburnum opulus bark;

e) between about 50 to about 150 mg per day of extract of Vitex agnus castus berry; and

f) between about 15 to about 75 mg per day by weight of citrus bioflavonoid(s).

In a more preferred embodiment, therapeutically effective amounts of each ingredient are as follows:

a) between about 240 to about 330 mg per day of gamma linolenic acid (GLA);

b) between about 375 to about 435 mg per day of alpha linolenic acid (ALA);

c) between about 30 to about 90 mg per day of vitamin E;

d) between about 135 to about 165 mg per day of extract of Viburnum opulus bark;

e) between about 60 to about 90 mg per day of extract of Vitex agnus castus berry; and

f) between about 30 to about 75 mg per day of citrus bioflavonoid(s).

For relief of the symptoms of PMS, the subject should preferably take a therapeutically effective amount of the composition daily through a sufficient number of menstrual cycles until satisfactory relief is attained. For relief of the symptoms of Irritable Bowel Syndrome or Interstitial Cystitis, the subject should preferably take a therapeutically effective amount of the composition daily until satisfactory relief of the symptoms is attained.

Preferably, the composition comprises the recommended daily dose of the ingredients a-f, as described above. Advantageously, the composition additionally comprises a suitable carrier. The composition can be administered once daily or alternatively, is subdivided into multiple doses to be administered during the course of a day. The composition is preferably subdivided into equally sized doses and more preferably into three equally sized doses.

Alternatively, ingredients a-f can be administered separately. For example, the ingredients can be administered as two or more separate compositions. Preferably, however, the ingredients are administered as a single composition, as described above.

As described above, the compositions of the present invention can optionally comprise additional ingredients which may be beneficial in the treatment of one of the aforesaid conditions. Vitamins, such as vitamin B6 are one example. When included typically between about 1 and about 200 mg per day and preferably between about 1 and about 10 mg per day of vitamin B6 is administered. Another example of an additional beneficial ingredient is a physiologically acceptable zinc salt. When included, typically between about 5 and about 100 mg per day, and preferably between about 5 and about 75 mg per day of the zinc salt is administered. Examples of physiologically acceptable salts of zinc include zinc sulfate, zinc citrate and zinc complexed or chelated with naturally occurring amino acids. As described above, these additional ingredients can be administered as part of a single daily dose or as part of subdivided doses which in combination provide the recommended daily amount of each ingredient.

The invention also provides a pharmaceutical pack or kit comprising one or more containers filled with one or more of the ingredients of the nutritional compositions of the invention. Optionally associated with such container(s) can be a notice in the form prescribed by a government agency regulating the manufacture, use or sale of pharmaceutical products, which notice reflects approval by the agency of manufacture, use of sale for human administration. The pack or kit can be labeled with information regarding mode of administration, sequence of administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a formulation or tablet. Agents assembled in a blister pack or other dispensing means is preferred.

### EXEMPLIFICATION

### Example 1

The Combination Composition of the Present Invention is Effective in Treating Premenstrual Symptoms

Design

Placebo controlled, double blind (patient and the physician were unaware if they were getting the medicine or the placebo), cross over design (every patient received the combination composition and placebo, hence served as her own control). One group of patients received the placebo formulation (1000 mg sunflower oil capsules, one capsule three times a day) for three months. The other group of patients received three times a day a soft gelatin capsule containing the following ingredients:

| | |
|---|---|
| Borage Oil | 450 mg supplying 90 to 108 mg GLA |
| Flax Oil | 250 mg supplying 125 to 145 mg ALA |
| Vitamin E | 30 I.U. |
| Viburnum opulus (Cramp bark) | 50 mg |
| extract | |
| Vitex agnus castus (Chaste Tree | 25 mg |
| berry) extract | |
| Orange bioflavonoids extract | 50 mg |

The patients were divided into two groups at random. At the end of three months, the patients were switched to treatment with the formulation (one capsule three times a day) for three months. The other group started with the formulation and after three months, was switched over to placebo treatment.

Patient Selection Criteria

Women between the age of 19 to 49 years who have recurring premenstrual symptoms were selected. All the patients were subjected to complete medical examination and medical history was recorded. Patients with PMS previously diagnosed by another physician were admitted to the study but they had to go through preliminary history and physical examination by the study gynecologist. The patients suffering from chronic or debilitating disease, with a history of a psychiatric condition that may confound the diagnosis and clinical course of PMS, unable to consistently record the symptoms and their intensity on the calendar, or using any prescription medication during the expected length of study were excluded from the study.

Number of Patients who completed the study

74 patients participated in the study. (38 subjects were on placebo for first three months followed by composition of the present invention for last three months and 36 patients were in reverse order.

Drop outs

8 subjects did not complete the study (2 subjects completed treatment with only placebo treatment while 4 subjects completed treatment only with the composition of the present invention) No medical reason for the drop outs was given.

Side Effects/Adverse Reactions: No cause effect relationship could be attributed

One patient: Fatigue, but the patient had history of mild iron deficiency anemia which may have caused the fatigue.

One patient: Gastrointestinal upset and exacerbation of hemorrhoids

One patient: Mid cycle transitory pain which started prior to commencement of treatment.

One patient: Sporadic episodes of premenstrual migraine which started prior to commencement of treatment.

Major Findings

The treatment group showed significant reduction in global scores. Global scores are defined as sum total of individual scores of all the 15 symptoms (listed in Table 1) monitored in the study. A large treatment benefit was observed for all the symptoms monitored in the study and listed in Table 1. For 9 out of 15 symptoms, the improvements increased from first cycle onwards (Table 2) while for the other 6 symptoms listed in Table 3, the benefits remain constant throughout the study period and there was no significant further improvement during subsequent cycles. Normally, a p values of less that or equal 0.05 is considered statistically significant. In our study, the p value was less than 0.001, indicating highly significant differences between the combination composition and placebo group. It is worth mentioning here that the mean percent reduction in global scores during the three cycles of treatment were 49.4%, 61.5%, and 67.1%, respectively. 46 patients reported 50% or higher reduction in global scores after first menstrual cycle and the number of such patients increased to 58 (78%) and 63 (85%) in the second and third cycle, respectively. The number of patients reporting 30% or better benefits is 63 (85%), 66 (89%), and 70 (95%) in cycle 1, 2, and 3, respectively. This clearly demonstrates the superiority of the formulation over the placebo.

TABLE 1
List of symptoms monitored during the study.

| | | |
|---|---|---|
| Cramps | 1 | Pre-menstrual cramps (last 7 days before menses) |
| GI problems | 2 | Nausea, diarrhea or any stomach or bowel |
| | | problem |
| Depression | 3 | Depression/crying easily/feeling down or hopeless |
| Anxiety | 4 | Anxiety/tension/feeling “on the edge” |
| Anger | 5 | Anger/irritability |
| Hopeless | 6 | Feeling hopeless or worthless or guilty |
| Alone | 7 | Wish to be alone |
| Concentration | 8 | Difficulty concentrating/less work done inside or |
| | | outside the house |
| Craving | 9 | Food cravings |
| Fatigue | 10 | Fatigue/less energy |
| Bloating | 11 | Bloating/swelling |
| Breast tenderness | 12 | Breast tenderness |
| Headache | 13 | Headache |
| Less sleep | 14 | Less sleep |
| Hot flushes | 15 | Hot Flushes |

For the following symptoms (Table 2), the improvement was observed from first cycle onwards and with the benefits of therapy increased in subsequent cycles.

TABLE 2
Mean difference between placebo and the treatment group scores.

| | | Cycle 1 | Cycle 2 | Cycle 3 |
|---|---|---|---|---|
| | | Mean | Mean | Mean |
| Symptom | p-value | Difference | Difference | Difference |
| Depression | <0.001 | 9.959 | 12.189 | 13.73 |
| Anxiety | <0.001 | 9.405 | 11.865 | 13.081 |
| Anger | <0.001 | 11.581 | 13.757 | 15.162 |
| Hopeless | 0.012 | 6.23 | 7.716 | 8.878 |
| Alone | 0.042 | 7.311 | 8.378 | 9.581 |
| Concentration | <0.001 | 9.041 | 11.27 | 12.554 |
| Craving | 0.003 | 8.486 | 10.068 | 11.689 |
| Fatigue | 0.008 | 10.419 | 12.919 | 13.189 |
| Bloating | 0.002 | 8.284 | 10.365 | 11.378 |

The following table (Table 3) lists the symptoms that improved from the first cycle but the improvements did not increase in subsequent cycles.

TABLE 3

| Symptom | Treatment p-value |
|---|---|
| Cramps (pre-menstrual, 7 days) | <0.001 |
| GI problems | <0.001 |
| Breast tenderness | <0.001 |
| Headache | <0.001 |
| Less sleep | <0.001 |
| Hot flushes | <0.001 |

Scores for each symptom for each cycle of the intervention are shown below in Table 4:

TABLE 4

| | Cycle 1 | | Cycle 2 | | Cycle 3 | |
|---|---|---|---|---|---|---|
| | Placebo | Treatment | Placebo | Treatment | Placebo | Treatmen |
| Cramps | 3.18 | 1.62 | 3.36 | 1.46 | 2.88 | 1.00 |
| GI-Problems (including | 9.49 | 4.64 | 9.50 | 4.01 | 9.45 | 3.35 |
| nausea, diarrhea, other | | | | | | |
| symptoms) | | | | | | |
| Depression/crying | 17.74 | 7.78 | 18.54 | 6.35 | 18.54 | 4.81 |
| easily/feeling hopeless | | | | | | |
| Anxiety/tension/feeling on the | 18.04 | 8.64 | 18.68 | 6.81 | 18.82 | 5.74 |
| edge | | | | | | |
| Anger/Irritability | 20.82 | 9.24 | 21.49 | 7.73 | 21.50 | 6.34 |
| Hopeless/worthless/guilty | 11.38 | 5.15 | 11.69 | 3.97 | 11.68 | 2.80 |
| feeling | | | | | | |
| Wish to be Alone | 13.30 | 5.99 | 13.54 | 5.16 | 13.42 | 3.84 |
| Difficulty in Concentration/less | 16.88 | 7.84 | 17.45 | 6.18 | 18.16 | 5.61 |
| work done in or out of house | | | | | | |
| Food Cravings | 16.65 | 8.16 | 16.80 | 6.73 | 17.34 | 5.65 |
| Fatigue/Less energy | 19.93 | 9.51 | 20.89 | 7.97 | 20.24 | 7.05 |
| Bloating/Swelling | 16.35 | 8.07 | 17.31 | 6.95 | 17.43 | 6.05 |
| Breast Tenderness | 16.05 | 7.99 | 16.74 | 6.45 | 16.00 | 5.92 |
| Headache | 9.38 | 3.59 | 10.20 | 3.20 | 10.08 | 2.47 |
| Less Sleep | 12.88 | 5.51 | 13.61 | 4.50 | 13.31 | 3.81 |
| Hot Flushes | 5.81 | 2.03 | 5.88 | 1.73 | 5.81 | 1.42 |

### Example 2

The Combination Composition of the Present Invention is Effective in Treating Irritable Bowel Syndrome and Interstitial Cystitis

One patient who participated in the clinical trial described in Example 1 reported additional benefits in controlling the symptoms of her Irritable Bowel Syndrome and Interstitial Cystitis while on this formulation.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating premenstrual symptoms in a subject in need of such treatment, said method comprising administering a therapeutically effective of ingredients a-f to the subject:
a) n-6 fatty acid or a source thereof;
b) n-3 fatty acid or a source thereof;
c) vitamin E;
d) extract of Viburnum opulus bark;
e) extract of Vitex agnus castus berry; and
f) citrus bioflavonoid(s).

2. The method of claim 1 wherein the subject is administered:
a) between about 45 to about 1000 mg per day of n-6 fatty acid;
b) between about 50 to about 1000 mg per day of the n-3 fatty acid;
c) between about 10 to about 90 mg per day of vitamin E;
d) between about 50 to about 600 mg per day of extract of Viburnum opulus bark;
e) between about 25 to about 300 mg per day of extract of Vitex agnus castus berry; and
f) between about 5 and about 100 parts mg per day of citrus bioflavonoid(s).

3. The method of claim 2 wherein ingredients a-f are administered as a composition.

4. The method of claim 3 the composition is administered to the subject one or more times a day.

5. The method of claim 4 wherein the n-6 fatty acid is gamma linolenic acid (GLA) and the n-3 fatty acid is alpha linolenic acid (ALA).

6. The method of claim 5 wherein the composition comprises fungal oil, borage oil, evening primrose oil or black current oil as the source of GLA.

7. The method of claim 6 wherein the composition comprises substantially pure GLA and/or ALA.

8. The method of claim 5 wherein the composition comprises flax oil or Perilla oil as the source of ALA.

9. The method of claim 5 the ratio of ALA to GLA in the composition is between about 1.0 to about 2.0 w/w.

10. The method of claim 3 wherein the ratio of n-6 fatty acid to n-3 fatty acid is about 1.75 to about 4.5 w/w.

11. The method of claim 3 wherein the composition additionally comprises vitamin B6 and/or a physiologically acceptable salt of zinc, wherein the subject is being administered between about 1 and about 200 mg per day of the vitamin B6 and/or between about 5 and about 100 mg per day of the zinc salt.

12. The method of claim 3 wherein the composition additionally comprises vitamin B6 and/or a physiologically acceptable salt of zinc, wherein the subject is being administered between about 1 and about 10 mg per day of vitamin B6 and/or between about 5 and about 75 mg per day of the zinc salt.

13. The method of claim 3 wherein vitamin E is in the form of d-alpha tocopherol, d-alpha tocopheryl acetate, mixed tocopherols, racemic tocopherols or tocotrienols.

14. The method of claim 4 wherein the subject is administered:
a) between about 200 to about 400 mg per day of gamma linolenic acid, (GLA);
b) between about 200 to about 500 mg per day of alpha linolenic acid (ALA);
c) between about 30 to about 90 mg per day of vitamin E;
d) between about 100 to about 300 mg per day of extract of Viburnum opulus bark;
e) between about 50 to about 150 mg per day of extract of Vitex agnus castus berry; and
f) between about 15 to about 75 mg per day by weight of citrus bioflavonoid(s).

15. A method of claim 4 wherein the subject is administered:
a) between about 240 to about 330 mg per day of gamma linolenic acid (GLA);
b) between about 375 to about 435 mg per day of alpha linolenic acid (ALA);
c) between about 30 to about 90 mg per day of vitamin E;
d) between about 135 to about 165 mg per day of extract of Viburnum opulus bark;
e) between about 60 to about 90 mg per day of extract of Vitex agnus castus berry; and
f) between about 30 to about 75 mg per day of citrus bioflavonoid(s).

16. The method of claim 15 wherein the composition additionally comprises vitamin B6 and/or a physiologically acceptable salt of zinc, wherein the subject is being administered between about 1 and about 10 mg per day of vitamin B6 and/or between about 5 and about 75 mg per day of the zinc salt.

17. The method of claim 15 wherein the composition additionally comprises a physiologically acceptable carrier.

18. The method of claim 17 wherein the physiologically acceptable carrier is a gelatin capsule, a foodstuff or beverage.

19. A method of treating premenstrual symptoms in a subject in need of such treatment, said method comprising administering three times daily a composition comprising:
a) between about 80 to about 110 mg per dose of gamma linolenic acid (GLA);
b) between about 125 to about 145 mg per dose of alpha linolenic acid (ALA);
c) between about 10 to about 30 mg per dose of vitamin E;
d) between about 45 to about 55 mg per dose of extract of Viburnum opulus bark;
e) between about 20 to about 30 mg per dose of extract of Vitex agnus castus berry; and
g) between about 10 to about 25 mg per dose of citrus bioflavonoid(s).

20. A composition comprising ingredients a-f:
a) n-6 fatty acid or a source thereof;
b) n-3 fatty acid or a source thereof;
c) vitamin E;
d) extract of Viburnum opulus bark;
e) extract of Vitex agnus castus berry; and
f) citrus bioflavonoid(s).

21. The composition of claim 20 wherein:
a) between about 45 to about 1000 parts of the total combined weight of ingredients a-f are gamma linolenic acid (GLA);
b) between about 50 to about 1000 parts of the total combined weight of ingredients a-f are alpha linolenic acid (ALA);
c) between about 10 to about 90 parts of the total combined weight of ingredients a-f are vitamin E;
d) between about 50 to about 600 parts of the total combined weight of ingredients a-f are extract of Viburnum opulus bark;
e) between about 25 to about 300 parts of the total combined weight of ingredients a-f are extract of Vitex agnus castus berry; and
f) between about 5 and about 100 parts of the total combined weight of ingredients a-f are citrus bioflavonoid(s).

22. The composition of claim 20 wherein:
a) between about 200 to about 400 parts of the total combined weight of ingredients a-f are gamma linolenic acid (GLA);
b) between about 200 to about 500 parts of the total combined weight of ingredients a-f are alpha linolenic acid (ALA);
c) between about 30 to about 90 parts of the total combined weight of ingredients a-f are vitamin E;
d) between about 100 to about 300 parts of the total combined weight of ingredients a-f are extract of Viburnum opulus bark;
e) between about 50 to about 150 parts of the total combined weight of ingredients a-f are extract of Vitex agnus castus berry; and
f) between about 15 to about 75 parts of the total combined weight of ingredients a-f are citrus bioflavonoid(s).

23. The composition of claim 20 wherein the composition comprises:
a) between about 45 to about 1000 mg of n-6 fatty acid;
b) between about 50 to about 1000 mg of n-3 fatty acid;
c) between about 10 to about 90 mg of vitamin E;
d) between about 50 to about 600 mg of extract of Viburnum opulus bark;
e) between about 25 to about 300 mg of extract of Vitex agnus castus berry; and
f) between about 5 and about 100 parts mg of citrus bioflavonoid(s).

24. The composition of claim 20 wherein the composition comprises:
a) between about 200 to about 400 mg of gamma linolenic acid, (GLA);
b) between about 200 to about 500 mg of alpha linolenic acid (ALA);
c) between about 30 to about 90 mg of vitamin E;
d) between about 100 to about 300 mg of extract of Viburnum opulus bark;
e) between about 50 to about 150 mg of extract of Vitex agnus castus berry; and
f) between about 15 to about 75 mg by weight of citrus bioflavonoid(s).

25. The composition of claim 20 wherein the composition comprises:
a) between about 240 to about 330 mg of gamma linolenic acid (GLA);
b) between about 375 to about 435 mg of alpha linolenic acid (ALA);
c) between about 30 to about 90 mg of vitamin E;
d) between about 135 to about 165 mg of extract of Viburnum opulus bark;
e) between about 60 to about 90 mg of extract of Vitex agnus castus berry; and
f) between about 30 to about 75 mg of citrus bioflavonoid(s).

26. The composition of claim 20, wherein the composition comprises:
a) between about 80 to about 110 mg of gamma linolenic acid (GLA);
b) between about 125 to about 145 mg of alpha linolenic acid (ALA);
c) between about 10 to about 30 mg of vitamin E;
d) between about 45 to about 55 mg of extract of Viburnum opulus bark;
e) between about 20 to about 30 mg of extract of Vitex agnus castus berry; and
f) between about 10 to about 25 mg of citrus bioflavonoid(s).

27. The composition of claim 25, additionally comprising a physiologically acceptable carrier.

28. The composition of claim 26, additionally comprising a physiologically acceptable carrier.

29. The composition of claim 27 wherein the carrier is a gelatin capsule.

30. The composition of claim 28 wherein the carrier is a gelatin capsule.

31. The composition of claim 27 wherein the carrier is a foodstuff or beverage.

32. The composition of claim 28 wherein the carrier is a foodstuff or beverage.

33. The composition of claim 22 wherein the n-6 fatty acid is gamma linolenic acid (GLA) and the n-3 fatty acid is alpha linolenic acid (ALA).

34. The composition of claim 33 wherein the composition comprises fungal oil, borage oil, evening primrose oil or black current oil as the source of GLA.

35. The composition of claim 34 wherein the composition comprises substantially pure GLA and/or ALA.

36. The composition of claim 33 wherein the composition comprises flax oil or Perilla oil as the source of ALA.

37. The composition of claim 33 the ratio of ALA to GLA in the composition is between about 1.0 to about 2.0 w/w.

38. The composition of claim 21 wherein the ratio of n-6 fatty acid to n-3 fatty acid is about 1.75 to about 4.5 w/w.

39. The composition of claim 21 wherein the composition additionally comprises vitamin B6 and/or a physiologically acceptable salt of zinc.

40. A method of treating irritable bowel symptoms or interstitial cystitis in a subject in need of such treatment, said method comprising the step of administering to the subject an effective amount of the composition of claim 19.
